(19) United States
(12) Patent Application Publication
(10) Pub. No.: US 2023/0202982 A1
(43) Pub. Date: Jun. 29, 2023

(54) 5-CHLORO-4-HYDROXY-1-METHYL-2-OXO-N-PHENYL-QUINOLINE-3-CARBOXAMIDE POTASSIUM SALT FOR TREATING INFLAMMATORY BOWEL DISEASES

(71) Applicant: Aqilion AB, Helsingborg (SE)

(72) Inventors: Martin Hans Johansson, Helsingborg (SE); David Crowe, Reading, Berkshire (GB)

(73) Assignee: Aqilion AB, Helsingborg (SE)

(21) Appl. No.: 17/922,072

(22) PCT Filed: Apr. 30, 2021
(86) PCT No.: PCT/EP2021/061464

§ 371 Date: Oct. 28, 2022

(30) Foreign Application Priority Data
Apr. 30, 2020 (GB) 2006390.5

(51) Int. Cl. C07D 215/56; A61P 1/04

(52) U.S. Cl. CPC C07D 215/56; A61P 1/04

(57) ABSTRACT

The invention provides the compound 5-chloro-4-hydroxy-1-methyl-2-oxo-N-phenyl-quinoline-3-carboxamide potassium salt. It is considered to have the structure of formula (I): The compound is provided in solid (eg crystalline or amorphous) or aqueous solution form. Pharmaceutical compositions comprising the compound are also provided. The compound finds use as a medicament, in particular for the treatment or prophylaxis of an inflammatory bowel disease.

### FIELD OF THE INVENTION

The present invention relates to the novel compound 5-chloro-4-hydroxy-1-methyl-2-oxo-N-phenyl-quinoline-3-carboxamide potassium salt. The compound finds particular use in the treatment and/or prophylaxis of inflammatory bowel diseases.

### BACKGROUND OF THE INVENTION

Inflammatory Bowel Diseases (IBDs) are a heterogeneous group of diseases and disorders that are characterised by chronic inflammation of the gastrointestinal wall. Symptoms caused by the chronic inflammation include abdominal pain, diarrhoea, general feeling of ill health, and loss of appetite and poor absorption of nutrients from food, which often leads to weight loss. There are two main forms of IBDs: ulcerative colitis (UC), which typically begins in the descending colon and rectum and may extend continuously to involve the entire colon (pancolitis), and Crohn’s disease (CD), which most commonly involves the ileum and ascending colon. Indeterminate Colitis (IC) may also be considered a form of IBD. An IBD is categorised as IC when the disease state is indistinguishable from CD and UC.

Currently-available treatments for IBDs are largely directed to the reduction of symptoms and the maintenance of remission. To prolong remission, long-term maintenance therapy is often required. First-line treatment often involves the use of aminosalicylates and/or corticosteroids. Second-line treatments include immunosuppressants, Tumor Necrosis Factor (TNF) inhibitors and integrin inhibitors. Second-line treatments may be used as a monotherapy, or in combination with one or more first or second-line treatments. Often, surgical intervention is required.

Immunomodulatory drugs have also shown promise for the treatment of IBDs. N-alkyl 1,2-dihydro-4-hydroxy-2-oxo-quinoline-3-carboxanilides (hereinafter referred to as N-alkyl quinoline-3-carboxanilides) are one particularly promising class of compounds that have been shown to have immunomodulatory properties. The immunomodulatory properties and therapeutic potential of N-alkyl quinoline-3-carboxanilides was first reported in the 1980s (for example, see US 4,547,511). One member of this class is laquinimod, which has been reported to be beneficial for the treatment of Crohn’s Disease (see for example, D′Haens et al., Gut. 2015, 64(8):1227-35, and WO 2011/014255).

Despite initial promise, poor efficacy, toxicity and instability have limited the clinical success of N-alkyl quinoline-3-carboxanilides. For example, Jansson et al., (J. Org. Chem. 2006, 71, 1658-1667) reported that N-alkyl quinoline-3-carboxanilides are chemically reactive towards nucleophiles, making them unstable in their neutral form. N-alkyl quinoline-3-carboxanilides, such as laquinimod, have also been shown to be readily metabolised by cytochrome P450 (CYPs) enzymes into various active metabolites, potentially having differing potencies, toxicities, and physicochemical properties (for example, see Tuvesson et al., 2005, Drug Metab. Dispos, 33:866-872, 2005). Notably, laquinimod was refused market authorisation in Europe for the treatment of relapsing remitting multiple sclerosis due to concerns over human safety and poor efficacy (EMA 2014 Public Assessment Report - EMA/451905/2014).

Unfortunately, for many patients, available treatments for IBDs are ineffective at reducing symptoms and slowing disease progression. Many of the available treatments also cause serious adverse effects, such as an increased risk of infections, inflammation of the liver, nausea and sickness, weight gain, and in rare cases progressive multifocal leukoencephalopathy. There is therefore a significant clinical need for more effective therapeutic and prophylactic treatments for IBDs. In particular, there is a need for effective treatments that provide clinical benefits for patient suffering from an IBD, whilst also displaying manageable side-effects.

### SUMMARY OF THE INVENTION

The present invention provides the compound 5-chloro-4-hydroxy-1-methyl-2-oxo-N-phenyl-quinoline-3-carboxamide potassium salt. The compound is considered to have the structure:

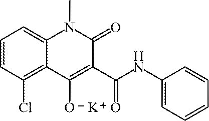 [SMILES: Cn1c(=O)c(C(=O)Nc2ccccc2)c([O-])c2c(Cl)cccc21 ; [K+]]

The present inventors have found that the compound according to the present invention is effective for the treatment and/or prophylaxis of inflammatory bowel diseases, such as Crohn’s disease and Ulcerative Colitis. Furthermore, the present inventors have surprisingly found that the compound according to the invention has especially beneficial physical properties; beneficial properties that provide significant advantages over the free acid compound 5-chloro-4-hydroxy-1-methyl-2-oxo-N-phenyl-quinoline-3-carboxamide.

For example, in solid form, the free acid 5-chloro-4-hydroxy-1-methyl-2-oxo-N-phenyl-quinoline-3-carboxamide exists as crystals in the form of long needles and those needles visibly agglomerate. On the other hand, 5-chloro-4-hydroxy-1-methyl-2-oxo-N-phenyl-quinoline-3-carboxamide potassium salt has been found by the present inventors in solid form to be powder-like crystals with a plate-like habit. The material is much easier to handle than the long needles of the free acid compound.

Further physico-chemical advantages of the 5-chloro-4-hydroxy-1-methyl-2-oxo-N-phenyl-quinoline-3-carboxamide potassium salt are described herein below.

The efficacy of 5-chloro-4-hydroxy-1-methyl-2-oxo-N-phenyl-quinoline-3-carboxamide potassium and its very favourable physicochemical properties allows for its use in effective therapeutic treatments for reducing the symptoms of inflammatory bowel diseases, in particular Crohn’s disease and Ulcerative Colitis, and prolonging disease remission.

The present invention further provides the compound of formula (I) for use as a medicament.

The present invention further provides the compound of formula (I) for use as a medicament in the treatment and/or prophylaxis of an inflammatory bowel disease.

The present invention also provides a pharmaceutical composition comprising the compound of formula (I) and at least one pharmaceutically acceptable excipient. The composition may optionally comprise one or more additional therapeutic agents.

The present invention further provides a method of treating and/or preventing an inflammatory bowel disease, said method comprising administering a pharmaceutically effective amount of the compound of formula (I) to a subject suffering from, or at risk of developing, an inflammatory bowel disease.

Also provided herein is a use of the compound of formula (I) in the manufacture of a medicament for the treatment and/or prophylaxis of an inflammatory bowel disease.

The present invention further provides a kit comprising the compound of formula (I), together with one or more pharmaceutically acceptable excipients, and optionally one or more further therapeutic agent. The kits of the present invention find use in the treatment and/or prophylaxis of inflammatory bowel diseases.

### DESCRIPTION OF FIGURES

FIG. 1 shows a DSC (Differential Scanning Calorimetry) trace for Example compound 1 (5-chloro-4-hydroxy-1-methyl-2-oxo-N-phenyl-quinoline-3-carboxamide potassium salt).

FIG. 2 shows an XRPD diffractogram for Example compound 1.

FIG. 3 shows optical microscope pictures of crystals of 5-chloro-4-hydroxy-1-methyl-2-oxo-N-phenyl-quinoline-3-carboxamide free acid (FIG. 3a) and of crystals of Example compound 1 (FIG. 3b).

FIG. 4 shows the change in body weight in groups of mice after treatment with Example compound 1 in a mouse model of inflammatory bowel disease.

FIGS. 5 and 6 show markers of inflammation in mice treated with Example compound 1 in a mouse model of inflammatory bowel disease.

FIG. 7 shows the level of Example compound 1 in the plasma at various time points in an in vivo pharmacokinetic study.

FIG. 8 shows the colitis score measured in mice in a DSS model of Ulcerative Colitis using C57B⅙ mice. Mice that received 5-chloro-4-hydroxy-1-methyl-2-oxoN-phenyl-quinoline-3-carboxamide free acid displayed a lower colitis score compared to mice that received vehicle only (CMC—Na, 2% w/v), or an anti-TNFα antibody.

FIG. 9 shows the body weight change (% change from day 1 of experiment) of C57B1/6 mice in the DSS mouse model of Ulcerative Colitis. Mice that received 5-chloro-4-hydroxy-1-methyl-2-oxo-N-phenyl-quinoline-3-carboxamide free acid displayed reduced weight loss compared to mice that received vehicle only (CMC—Na, 2% w/v), or an anti-TNFα antibody.

FIG. 10 shows the colon length (cm) in mice in the C57B1/6 DSS mouse model of Ulcerative Colitis at Day 10 of the experiment. Mice that received 5-chloro-4-hydroxy-1-methyl-2-oxo-N-phenyl-quinoline-3-carboxamide free acid were found to have greater colon length compared to mice that received vehicle only (CMC—Na, 2% w/v), or an anti-TNFα antibody.

### DETAILED DESCRIPTION OF THE INVENTION

The present inventors have found that 5-chloro-4-hydroxy-1-methyl-2-oxo-N-phenylquinoline-3-carboxamide potassium salt is effective for the treatment and/or prophylaxis of inflammatory bowel diseases, such as Crohn’s disease and Ulcerative Colitis. Furthermore, the present inventors have surprisingly found that the compound according to the invention has especially beneficial physical properties; beneficial properties that provide significant advantages over the free acid compound 5-chloro-4-hydroxy-1-methyl-2-oxo-N-phenyl-quinoline-3-carboxamide.

### Advantageous Crystalline Form

In solid form, the free acid 5-chloro-4-hydroxy-1-methyl-2-oxo-N-phenyl-quinoline-3-carboxamide forms crystals and those crystals are long needles which are seen to agglomerate. On the other hand, 5-chloro-4-hydroxy-1-methyl-2-oxo-N-phenylquinoline-3-carboxamide potassium salt has been found by the present inventors in solid form to be powder-like crystals with a plate-like habit. The material is much easier to handle than the long needles of the free acid compound.

5-chloro-4-hydroxy-1-methyl-2-oxo-N-phenyl-quinoline-3-carboxamide potassium salt has been found to generally have a particle size distribution (PSD) of under 10 microns, significantly smaller than the needle length of 200-300 microns for 5-chloro-4-hydroxy-1-methyl-2-oxo-N-phenyl-quinoline-3-carboxamide free acid. The PSD for the potassium salt is also advantageously smaller than for other salt forms; for example, both the sodium and the lithium salt were found to have PSDs of 10 to 20 microns.

The invention therefore further provides a compound of Formula (I) having D50 size in the range 0.5 to 7 µm and a D90 size in the range 5 to 10 µm. Preferably, the compound has a D50 size in the range 2 to 5 µm and a D90 size in the range 7 to 9 µm.

The current inventors have further established that 5-chloro-4-hydroxy-1-methyl-2-oxo-N-phenyl-quinoline-3-carboxamide potassium salt has better thermal stability than 5-chloro-4-hydroxy-1-methyl-2-oxo-N-phenyl-quinoline-3-carboxamide free acid. Whereas the free acid melts at 178° C. and degrades at 260° C., 5-chloro-4-hydroxy-1-methyl-2-oxo-N-phenyl-quinoline-3-carboxamide potassium salt melts at 347° C. and also only degrades at that temperature. The increased thermal stability brings helpful flexibility to manufacturing processes, and also allows for the compound to have a longer shelf life.

In a differential scanning calorimetry (DSC) analysis, there was no significant endotherm before the melt with onset at ~ 343° C. The maximum endothermic heat flow was seen at 361.3° C.

The invention therefore provides the compound of formula (I) in crystalline form wherein the crystalline form is characterized by a differential scanning calorimetry trace recorded at a heating rate of 10° C. per minute which shows a maximum in endothermic heat flow with a peak at 361.3±2° C.

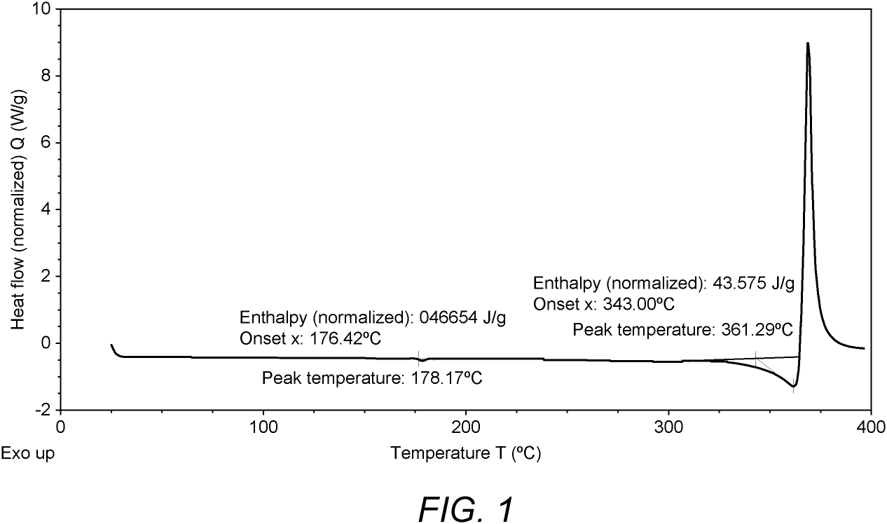

For example, the crystalline form may be characterized by a differential scanning calorimetry trace substantially in accordance with that shown in FIG. 1.

A further advantageous property of 5-chloro-4-hydroxy-1-methyl-2-oxo-N-phenylquinoline-3-carboxamide potassium salt is it exits in a non-hydrated form and that it is not hygroscopic.

A preferred solid form of 5-chloro-4-hydroxy-1-methyl-2-oxo-N-phenyl-quinoline-3-carboxamide potassium salt is a crystalline salt having a powder X-ray diffractogram with characteristic peaks at 2θ = 6.9±0.2°, 15.6±0.2°, 24.9±0.2° and 28.4±0.2°. The powder X-ray diffractogram may have further characteristic peaks at 2θ = 20.9°±0.2, 24.2±0.2°, 25.2±0.2°, 25.6±0.2° and 27.3±0.2°. The powder X-ray diffractogram may have further characteristic peaks at 2θ = 13.9±0.2°, 17.8±0.2°, 23.6±0.2° 26.8±0.2°, 29.2±0.2° and 34.0±0.2°.

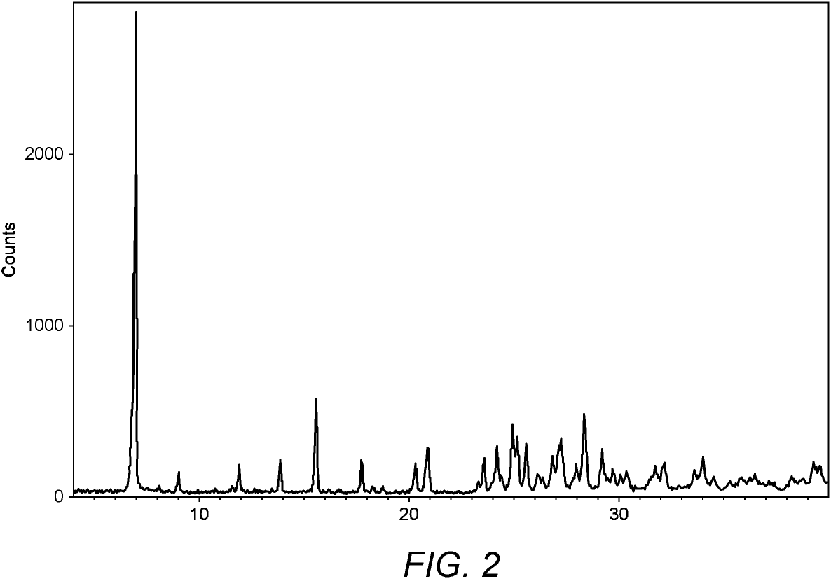

An example XRPD trace for the crystalline salt is shown in FIG. 2. The invention therefore provides the compound of formula (I) in crystalline form wherein the crystalline form is characterized by a powder X-ray diffraction pattern in which the peak positions are substantially in accordance with the peak positions of the pattern shown in FIG. 2.

### Beneficial Formation of Crystalline Form

The present inventors have surprisingly found that 5-chloro-4-hydroxy-1-methyl-2-oxo-N-phenyl-quinoline-3-carboxamide potassium salt can be provided in crystalline form from a broad range of solvents. Crystals of the same type were formed when the compound was crystallised from any of ethanol, acetone, ethyl acetate, acetonitrile, THF, toluene, methanol/water 50:50 and iso-propanol/water 90:10. The fact that crystals can consistently be formed from a wide variety of solvents provides helpful flexibility for manufacturing processes. Other salts that have been investigated do not display such flexibility in different solvents. For the parent free acid compound, it has been found that at least two polymorphic forms exist and that the form that is produced depends on the crystallisation solvent.

The invention accordingly provides a method of preparing 5-chloro-4-hydroxy-1-methyl-2-oxo-N-phenyl-quinoline-3-carboxamide potassium salt by crystallising from a solvent or solvent mixture wherein the solvent is selected from any one or more of ethanol, acetone, ethyl acetate, acetonitrile, THF, toluene, methanol/water 50:50, iso-propanol/water 90:10 and iso-propanol. Preferred solvents include ethanol, acetone, ethyl acetate, acetonitrile, iso-propanol/water 90:10 and iso-propanol.

5-chloro-4-hydroxy-1-methyl-2-oxo-N-phenyl-quinoline-3-carboxamide may be prepared using methods known to those skilled in the art of organic chemistry. Specific methods for preparing 5-chloro-4-hydroxy-1-methyl-2-oxo-N-phenylquinoline-3-carboxamide and its potassium salt according to the invention are described herein in the Examples section.

### Solubility in Aqueous Solvents

An important feature for any drug compound is its solubility in water: it determines how readily the compound can be administered in solution; it also determines how well the compound can be absorbed into the body of the patient and distributed around the body. It thus has a significant effect on the bioavailability of the drug.

It has been found that 5-chloro-4-hydroxy-1-methyl-2-oxo-N-phenyl-quinoline-3-carboxamide free acid has relatively low solubility. For example, in PBS (pH 7.4), 5-chloro-4-hydroxy-1-methyl-2-oxo-N-phenyl-quinoline-3-carboxamide free acid is so sparingly soluble as to be not quantifiable. On the other hand, the present inventors have found that 5-chloro-4-hydroxy-1-methyl-2-oxo-N-phenyl-quinoline-3-carboxamide potassium salt dissolves to 0.91 µg/ml. Similarly, in simulated colonic fluid (SCF), 5-chloro-4-hydroxy-1-methyl-2-oxo-N-phenyl-quinoline-3-carboxamide potassium salt is over twice as soluble (7.99 µg/ml compared with 3.44 µg/ml).

The inventors have further demonstrated in an in vivo study that 5-chloro-4-hydroxy-1-methyl-2-oxo-N-phenyl-quinoline-3-carboxamide potassium salt is absorbed through the intestine into the plasma in rats.

This beneficial increased solubility provides an advantage to 5-chloro-4-hydroxy-1-methyl-2-oxo-N-phenyl-quinoline-3-carboxamide potassium salt in dissolved form.

A further advantageous property of 5-chloro-4-hydroxy-1-methyl-2-oxo-N-phenylquinoline-3-carboxamide potassium salt is that it is stable in aqueous solution. It is also stable when in a slurry (beyond its solubility limit) in water.

Accordingly, the invention provides an aqueous solution comprising 5-chloro-4-hydroxy-1-methyl-2-oxo-N-phenyl-quinoline-3-carboxamide potassium salt in water. The aqueous solution may, for example, be buffered. For example, a buffered solution according to the invention may be a solution in phosphate-buffered saline (PBS). A solution according to the invention may be an aqueous pharmaceutical composition.

The compound of formula (I) may also be prepared in solid, non-crystalline form. Such forms are also known as amorphous.

### Clinical Efficacy

The compound of the invention shows a surprising efficacy in treating or preventing the symptoms and development of inflammatory bowel diseases. In particular, the present inventors have found that the compound according to formula (I) displays surprisingly beneficial properties for the treatment or prophylaxis of IBDs, such as Crohn’s disease and Ulcerative Colitis.

As discussed in more detail below, the present inventors have found that 5-chloro-4-hydroxy-1-methyl-2-oxo-N-phenyl-quinoline-3-carboxamide potassium salt was effective in inhibiting inflammation/edema associated with inflammatory bowel disease, as evaluated in a CD4+ adoptive transfer induced inflammatory bowel disease in mice. Furthermore, mice treated orally with 5-chloro-4-hydroxy-1-methyl-2-oxo-N-phenyl-quinoline-3-carboxamide potassium salt had a significant increase in CYP1A1 mRNA expression compared to untreated animals indicating that the compound is effective in activating the aryl hydrocarbon receptor (AhR) in the colon.

The inventors have further found that 5-chloro-4-hydroxy-1-methyl-2-oxo-N-phenylquinoline-3-carboxamide reduced weight loss and had a protective effect against the development of the clinical symptoms of Ulcerative Colitis in an in vivo mouse model of Ulcerative Colitis. The inventors also found that 5-chloro-4-hydroxy-1-methyl-2-oxo-N-phenyl-quinoline-3-carboxamide was effective at reducing colon shortening which indicates a reduction in edema associated with Ulcerative Colitis. The surprising efficacy of DELAQ allows for its use in effective treatment and prophylaxis of IBDs, such as Crohn’s disease and Ulcerative Colitis.

The invention thus provides the compound of formula (I) for use as a medicament.

The present invention further provides the compound of formula (I) for use as a medicament in the treatment and/or prophylaxis of an inflammatory bowel disease.

The present invention further provides a method of treating and/or preventing an inflammatory bowel disease, said method comprising administering a pharmaceutically effective amount of the compound of formula (I) to a subject suffering from, or at risk of developing, an inflammatory bowel disease.

Also provided herein is a use of the compound of formula (I) in the manufacture of a medicament for the treatment and/or prophylaxis of an inflammatory bowel disease.

N-desalkyl quinoline-3-carboxanilides have previously been reported to be active metabolites of N-alkyl quinoline-3-carboxanilides such as laquinimod and tasquinimod. In isolated form, N-desalkyl quinoline-3-carboxanilides have been reported to be unsuitable for in vivo administration due to poor stability and low aqueous solubility (for example, see Tuvesson et al., 2005, Drug Metab. Dispos, 33:866-872, 2005, WO 2012/050500 and Mariout et al., 2017, Tox. Appl. Pharm., 326, 54-65).

The present inventors have found that 5-chloro-4-hydroxy-1-methyl-2-oxo-N-phenylquinoline-3-carboxamide, and in particular 5-chloro-4-hydroxy-1-methyl-2-oxo-N-phenyl-quinoline-3-carboxamide potassium salt, is surprisingly effective at treating and preventing IBDs, in particular CD and UC, and has good stability and aqueous solubility.

### Pharmaceutical Compositions

While it is possible for a compound of formula (I) to be administered alone, it is preferable for it to be present in a composition and particularly in a pharmaceutical composition. Pharmaceutical compositions of the present invention comprise a compound of formula (I) and one or more pharmaceutically acceptable excipient.

Pharmaceutical compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The compound of formula (I) may also be presented as a bolus, electuary or paste. Various pharmaceutically acceptable carriers and their formulation are described in standard formulation treatises, e.g., Remington’s Pharmaceutical Sciences by E. W. Martin. See also Wang, Y. J. and Hanson, M. A., Journal of Parenteral Science and Technology, Technical Report No. 10, Supp. 42:2S, 1988.

Pharmaceutical compositions include those suitable for oral, parenteral (including subcutaneous, intradermal, intraosseous infusion, intramuscular, intravascular (bolus or infusion), and intramedullary), intraperitoneal, transmucosal, transdermal, rectal and topical (including dermal, buccal, sublingual and intraocular) administration although the most suitable route may depend upon for example the type of IBD that is under treatment.

Pharmaceutical compositions for rectal administration may be presented as a suppository with carriers such as cocoa butter, synthetic glyceride esters or polyethylene glycol. Such carriers are typically solid at ordinary temperatures, but liquefy and/or dissolve in the rectal cavity to release the drug.

Certain compounds are known that, under suitable conditions, for example in the human body, can be converted into 5-chloro-4-hydroxy-1-methyl-2-oxo-N-phenylquinoline-3-carboxamide by de-alkylation or by hydrolysis. A known compound that can be converted in that way is 5-chloro-N-ethyl-4-hydroxy-1-methyl-2-oxo-N phenyl-1,2-dihydroquinoline-3-carboxamide (known as laquinimod):

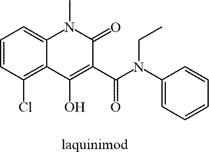

Preferably, compositions of the invention contain laquinimod at an amount of less than 10 mole percent (mol%) of the total combined number of moles of 5-chloro-4-hydroxy-1-methyl-2-oxo-N-phenyl-quinoline-3-carboxamide potassium salt and laquinimod present in the composition. More preferably, compositions comprising 5-chloro-4-hydroxy-1-methyl-2-oxo-N-phenyl-quinoline-3-carboxamide potassium salt contain laquinimod at amount of less than 5 mol%. For example, less than 4, 3, 2 or 1 mol% (for example, less than 0.9, 0.8, 0.7, 0.6, 0.5, 0.4, 0.3, 0.2 or 0.1 mol%) of laquinimod. Even more preferably, the composition of the invention is substantially free from laquinimod.

Preferably, compositions of the invention comprising 5-chloro-4-hydroxy-1-methyl-2-oxo-N-phenyl-quinoline-3-carboxamide potassium salt, contain laquinimod at an amount of less than 10 wt% of the total combined mass of 5-chloro-4-hydroxy-1-methyl-2-oxo-N-phenyl-quinoline-3-carboxamide potassium salt and laquinimod present in the composition. More preferably, laquinimod is present in the composition of the invention at an amount of less than 5 wt%. For example, less than 4, 3, 2 or 1 wt% (for example, less than 0.9, 0.8, 0.7, 0.6, 0.5, 0.4, 0.3, 0.2 or 0.1 wt%) of laquinimod.

Even more preferably, the composition of the invention is substantially free from laquinimod.

The compositions of the invention may comprise one or more further therapeutic agents. Examples of further therapeutic agents that may be present in a composition of the present invention include, but not limited to, aminosalicylates (for example, mesalazine, olsalazine, sulphasalazine, balsalazide), corticosteroids (for example, prednisolone, prednisone, methylprednisolone, budesonide, hydrocortisone and beclometasone dipropionate), immunosuppressants (for example, azathioprine, mercaptopurine, methotrexate, ciclosporin and tacrolimus), anti-TNF drugs (for example, infliximab, adalimumab and golimumab), antibiotics (for example, ciprofloxacin and metronidazole), anti-integrin drugs (for example, vedolizumab and natalizumab), interleukin inhibitors (for example, ustekinumab) Janus Kinase inhibitors (for example, tofacitinib, filgotinib, upadacitinib, and TYK2 inhibitors such as BMS-986165).

### Inflammatory Bowel Diseases

The compound of formula (I), and pharmaceutical compositions of the invention, find use in treating IBDs, for example CD and UC.

As such, the compound of formula (I) according to the invention, or composition of the invention, may be administered to a subject having an IBD, such as CD or UC. The subject may be a human subject, for example a human patient.

The subject may have an IBD that may be classed as refractory, relapsed or refractory-relapsed. For example, the subject may have refractory, relapsed or refractory-relapsed CD or UC. Additionally, or alternatively, the subject may have an IBD that is partially or completely resistant to established IBD treatments, such as aminosalicylates and corticosteroids. For example, the IBD may be CD or UC that is partially or completely resistant to aminosalicylate and/or corticosteroid treatment or prophylaxis. Additionally, or alternatively, the subject may be one who has experienced, or at risk of experiencing, an adverse reaction to an established IBD treatment, such as aminosalicylates and corticosteroids.

The compound of formula (I) according to the invention, and compositions of the invention, may be administered to a subject known or suspected of being at risk of developing an IBD. For example, subjects with a known or suspected genetic predisposition for developing an IBD, such as CD or UC. For example, the compound of formula (I), or composition of the invention, may be administered to a subject in need of extended remission of an IBD and/or slower progression of an IBD.

The compound of formula (I) and compositions of the invention find utility in a method of treating or preventing an IBD, said method comprising a step of administering the compound of formula (I), or a composition of the invention, to a subject having an IBD, such as CD or UC. In certain embodiments, the method of treating or preventing an IBD comprises a step of administering the compound of formula (I), or a composition of the invention, to a subject known or suspected of being at risk of developing an IBD.

In certain embodiments, the method of treatment or prophylaxis comprises a step of delivering the compound of formula (I), or a pharmaceutical composition of the invention, to the small and/or large intestine of a subject. For example, a step of delivering the compound of formula (I), or a pharmaceutical composition of the invention, to one or more of the duodenum, jejunum and ileum; and/or one or more of the caecum, ascending colon, traverse colon, descending colon and/or sigmoid colon. The method of treatment or prophylaxis may also comprise a step of orally or rectally administering the compound of formula (I), or a composition of the invention, to a subject.

The compound of formula (I) also finds use in the manufacture of a medicament for the treatment or prophylaxis of an IBD. For example, the compound of formula (I) may be used in the manufacture of a medicament for the treatment or prophylaxis of CD or UC.

### Delivery to the Small and/or Large Intestine

The composition according to the invention may be adapted for selective release of the compound of formula (I) in the small intestine or the large intestine following rectal or oral administration. For example, in certain embodiments, the compound of formula (I), or pharmaceutical composition of the invention, is administered locally to the small and/or large intestine. This may be accomplished by the use of particular coatings and/or formulations.

The compositions of the invention may have an enteric coating. Enteric coatings which protect the active ingredients in a composition from attack and degradation in the stomach, and permit release within the intestines, are known. The optimal coating for any particular formulation depends on the exact intended use, and coatings may be tailored to release the active ingredient in a particular region of the intestines, or at a particular time following ingestion.

The composition of the invention may be adapted to release the compound of formula (I) in the small intestine, for example, in one or more of the duodenum, jejunum and ileum. Additionally, or alternatively, the composition of the invention may be adapted to release the compound of formula (I) in the large intestine, for example, in one or more of the caecum, ascending colon, traverse colon, descending colon and/or sigmoid colon.

The composition of the invention may be in a solid or semi-solid form, preferably comprising an enteric coating, adapted to release the compound of formula (I) in the small intestine and/or large intestine. Such a formulation may contain one or more intermediate layers between the active ingredient and the outer enteric coating. In certain embodiments, it is possible for a composition of the invention to release a portion of its contents at one or more particular regions of the small intestine, and a further portion of its contents in one or more particular regions of the large intestine.

### Dosage Regimens

The amount of the compound of formula (I) which is required to achieve a therapeutic effect will vary with particular route of administration and the characteristics of the subject under treatment, for example the species, age, weight, sex, medical conditions, the particular IBD and its severity, and other relevant medical and physical factors.

An ordinarily skilled physician can readily determine and administer an effective amount of the compound of formula (I) required for treatment or prophylaxis of the IBD.

The compound of formula (I) may be administered daily (including several times daily), every second or third day, weekly, every second, third or fourth week or even as a high single dose depending on the subject and IBD to be treated.

Preferably, the compound of formula (I) (excluding the mass of any counterion or solvent), may be administered in an amount of about 1 to 1000 mg per administration. For example, 1, 5, 10, 15, 20, 25, 40, 50, 60, 70, 80, 90, 100, 110, 120, 130, 140, 150, 200, 300, 400, 500, 600, 700, 800, 900 and 1000 mg.

In certain embodiments, the compound of formula (I) is administered as a composition. Preferably, the composition is a pharmaceutical composition of the present invention.

Whilst the compound of formula (I) may be used as the sole active ingredient in the present invention, it is also possible for it to be used in combination with one or more further therapeutic agent(s), and the use of such combinations provides one embodiment of the invention. Such further therapeutic agents may be agents useful in the treatment or prophylaxis of an IBD, or other pharmaceutically active materials. Such agents are known in the art. Examples of further therapeutic agents for use in the present invention include those described herein.

The one or more further therapeutic agent(s) may be used simultaneously, sequentially or separately with/from the administration of the dosage the compound of formula (I). The individual components of such combinations can be administered separately at different times during the course of therapy or concurrently in divided or single combination forms. An ordinarily skilled physician can readily determine and administer the effective amount of one or more therapeutic agent required to have the desired therapeutic effect.

The compound of formula (I), or may be administered as an oral or rectal dosage, and thus the dosage of the compound of formula (I) must be in the form suitable for delivery of the compound of formula (I) to the small and/or large intestine.

Preferred unit dosage compositions for use according to the invention are those containing an effective dose, or an appropriate fraction thereof, of the compound of formula (I). The release of the compound of formula (I) from certain composition may also be sustained, for example, if the composition contains suitable controlled-release excipients.

### Kits

The present invention provides a kit comprising the compound of formula (I), one or more pharmaceutically acceptable excipients, and optionally one or more further therapeutic agents that are useful in the treatment or prophylaxis of an IBD. Examples of such further therapeutic agents include those described herein as being suitable for use in the present invention, and being optionally present in a pharmaceutical composition of the invention as a further therapeutic agent.

Kits of the present invention find use in the treatment and prophylaxis of an IBD, especially CD and UC.

For the avoidance of doubt, the compound of formula (I) present in a kit according to the present invention is in a form and quantity suitable for use according to the present invention. Suitable pharmaceutical compositions and formulations are described herein. The skilled person can readily determine a quantity of the compound of formula (I) suitable for including in a kit of the invention, and for use according the invention.

### Further Aspects of the Invention

The current inventors also provide 5-chloro-4-hydroxy-1-methyl-2-oxo-N-phenylquinoline-3-carboxamide sodium salt. The compound is considered to have the structure:

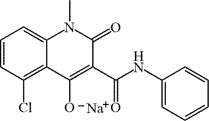

The present inventors have found that the compound of formula (II) is effective for the treatment and/or prophylaxis of inflammatory bowel diseases, such as Crohn’s disease and Ulcerative Colitis. Furthermore, the present inventors have surprisingly found that the compound of formula (II) has especially beneficial physical properties; beneficial properties that provide significant advantages over the free acid compound 5-chloro-4-hydroxy-1-methyl-2-oxo-N-phenyl-quinoline-3-carboxamide.

For example, in solid form, the free acid 5-chloro-4-hydroxy-1-methyl-2-oxo-N-phenyl-quinoline-3-carboxamide exists as crystals in the form of long needles and those needles visibly agglomerate. On the other hand, 5-chloro-4-hydroxy-1-methyl-2-oxo-N-phenyl-quinoline-3-carboxamide sodium salt has been found by the present inventors in solid form to be powder-like crystals with a plate-like habit. The material is much easier to handle than the long needles of the free acid compound.

Further physico-chemical advantages of the 5-chloro-4-hydroxy-1-methyl-2-oxo-N-phenyl-quinoline-3-carboxamide sodium salt are described herein below.

The efficacy of 5-chloro-4-hydroxy-1-methyl-2-oxo-N-phenyl-quinoline-3-carboxamide sodium and its favourable physicochemical properties allows for its use in effective therapeutic treatments for reducing the symptoms of inflammatory bowel diseases, in particular Crohn’s disease and Ulcerative Colitis, and prolonging disease remission.

The present invention further provides the compound of formula (II) for use as a medicament.

The present invention further provides the compound of formula (II) for use as a medicament in the treatment and/or prophylaxis of an inflammatory bowel disease.

The present invention also provides a pharmaceutical composition comprising the compound of formula (II) and at least one pharmaceutically acceptable excipient. The composition may optionally comprise one or more additional therapeutic agents.

The present invention further provides a method of treating and/or preventing an inflammatory bowel disease, said method comprising administering a pharmaceutically effective amount of the compound of formula (II) to a subject suffering from, or at risk of developing, an inflammatory bowel disease.

Also provided herein is a use of the compound of formula (II) in the manufacture of a medicament for the treatment and/or prophylaxis of an inflammatory bowel disease. The present invention further provides a kit comprising the compound of formula (II), together with one or more pharmaceutically acceptable excipient, and optionally one or more further therapeutic agent. The kits of the present invention find use in the treatment and/or prophylaxis of inflammatory bowel diseases.

The current inventors have established that 5-chloro-4-hydroxy-1-methyl-2-oxo-N-phenyl-quinoline-3-carboxamide sodium salt has better thermal stability than 5-chloro-4-hydroxy-1-methyl-2-oxo-N-phenyl-quinoline-3-carboxamide free acid. Whereas the free acid melts at 178° C. and degrades at 260° C., 5-chloro-4-hydroxy-1-methyl-2-oxo-N-phenyl-quinoline-3-carboxamide sodium salt melts at 356° C. and also only degrades at around 350° C. The increased thermal stability brings helpful flexibility to manufacturing processes, and also allows for the compound to have a longer shelf life.

A preferred solid form of 5-chloro-4-hydroxy-1-methyl-2-oxo-N-phenyl-quinoline-3-carboxamide sodium salt is a crystalline salt having a powder X-ray diffractogram with characteristic peaks at 2θ = 7.2±0.2°, 24.5±0.2°, 26.4±0.2° and 26.5±0.2°. The powder X-ray diffractogram may have further characteristic peaks at 2θ = 9.3±0.2°, 15.9±0.2°, 23.1±0.2° and 25.2±0.2°. The powder X-ray diffractogram may have further characteristic peaks at 2θ = 12.2±0.2°, 18.9±0.2°, 22.2±0.2° and 29.7±0.2°.

The current inventors also provide 5-chloro-4-hydroxy-1-methyl-2-oxo-N-phenylquinoline-3-carboxamide lithium salt. The compound is considered to have the structure:

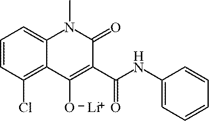 [SMILES: Cn1c(=O)c(C(=O)Nc2ccccc2)c([O-])c2c(Cl)cccc21 ; [Li+]]

The present inventors have found that the compound of formula (III) is effective for the treatment and/or prophylaxis of inflammatory bowel diseases, such as Crohn’s disease and Ulcerative Colitis. Furthermore, the present inventors have surprisingly found that the compound of formula (III) has especially beneficial physical properties; beneficial properties that provide significant advantages over the free acid compound 5-chloro-4-hydroxy-1-methyl-2-oxo-N-phenyl-quinoline-3-carboxamide.

For example, in solid form, the free acid 5-chloro-4-hydroxy-1-methyl-2-oxo-N-phenyl-quinoline-3-carboxamide exists as crystals in the form of long needles and those needles visibly agglomerate. On the other hand, 5-chloro-4-hydroxy-1-methyl-2-oxo-N-phenyl-quinoline-3-carboxamide lithium salt has been found by the present inventors in solid form to be powder-like crystals with a plate-like habit. The material is much easier to handle than the long needles of the free acid compound.

Further physico-chemical advantages of the 5-chloro-4-hydroxy-1-methyl-2-oxo-N-phenyl-quinoline-3-carboxamide lithium salt are described herein below.

The efficacy of 5-chloro-4-hydroxy-1-methyl-2-oxo-N-phenyl-quinoline-3-carboxamide lithium salt and its favourable physicochemical properties allows for its use in effective therapeutic treatments for reducing the symptoms of inflammatory bowel diseases, in particular Crohn’s disease and Ulcerative Colitis, and prolonging disease remission.

The present invention further provides the compound of formula (III) for use as a medicament.

The present invention further provides the compound of formula (III) for use as a medicament in the treatment and/or prophylaxis of an inflammatory bowel disease.

The present invention also provides a pharmaceutical composition comprising the compound of formula (III) and at least one pharmaceutically acceptable excipient. The composition may optionally comprise one or more additional therapeutic agents.

The present invention further provides a method of treating and/or preventing an inflammatory bowel disease, said method comprising administering a pharmaceutically effective amount of the compound of formula (III) to a subject suffering from, or at risk of developing, an inflammatory bowel disease.

Also provided herein is a use of the compound of formula (III) in the manufacture of a medicament for the treatment and/or prophylaxis of an inflammatory bowel disease. The present invention further provides a kit comprising the compound of formula (III), together with one or more pharmaceutically acceptable excipient, and optionally one or more further therapeutic agent. The kits of the present invention find use in the treatment and/or prophylaxis of inflammatory bowel diseases.

The current inventors have established that 5-chloro-4-hydroxy-1-methyl-2-oxo-N-phenyl-quinoline-3-carboxamide lithium salt has better thermal stability than 5-chloro-4-hydroxy-1-methyl-2-oxo-N-phenyl-quinoline-3-carboxamide free acid. Whereas the free acid melts at 178° C. and degrades at 260° C., 5-chloro-4-hydroxy-1-methyl-2-oxo-N-phenyl-quinoline-3-carboxamide lithium salt melts at 362° C. and also only degrades at around 360° C. The increased thermal stability brings helpful flexibility to manufacturing processes, and also allows for the compound to have a longer shelf life.

A preferred solid form of 5-chloro-4-hydroxy-1-methyl-2-oxo-N-phenyl-quinoline-3-carboxamide lithium salt is a crystalline salt having a powder X-ray diffractogram with characteristic peaks at 2θ = 6.7±0.2°, 21.2±0.2°, 24.3±0.2° and 25.9±0.2°. The powder X-ray diffractogram may have further characteristic peaks at 2θ = 10.4±0.2°, 15.8±0.2°, 27.0±0.2° and 28.6±0.2°. The powder X-ray diffractogram may have further characteristic peaks at 2θ = 19.8±0.2°, 20.0±0.2°, and 30.6±0.2°.

### Equivalents

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic documents.

The following Examples illustrate the invention.

### EXAMPLES

### Example 1: Synthesis of 5-chloro-4-hydroxy-1-methyl-2-oxo-N-phenyl-quinoline-3-carboxamide Potassium Salt (Example Compound 1)

### Step A) Synthesis of 5-chloro-4-hydroxy-1-methyl-2-oxo-N-phenyl-quinoline-3-carboxamide Free Acid

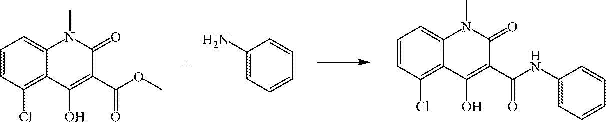

A mixture of methyl 5-chloro-4-hydroxy-1-methyl-2-oxo-quinoline-3-carboxylate (25 g, 0.0934 mol) and aniline (17.4 g, 0.0333 mol, 2 eq) in Toluene (600 mL) were stirred at 100° C. for 17h. HPLC revealed total conversion to product. The reaction was removed from heating and the product precipitated. The reaction was left to stand at room temperature for two days. The soft solid cake was suspended in n-heptane (500 mL) and after 5 minutes of stirring the solid was filtered off. The solid was washed with a 1:1 mixture of toluene and n-heptane (1000 mL) to give crude 5-chloro-4-hydroxy-1-methyl-2-oxo-N-phenyl-quinoline-3-carboxamide. The product was recrystallized from toluene and washed with heptane and then further purified by column chromatography (petroleum ether 100% -> DCM 100%). Final recrystallization from AcCN gave the desired product (21.5 g, 70% yield).

LC/MS: M+H=329.10. ¹H NMR (400 MHz, DMSO-d₆) δ: 12.75 (1H, s), 7.73 (1H, m), 7.65 (3H, m), 7.44 (3H, m), 7.22 (1H, m), 3.70 (s, 3H).

### Step B) Preparation of Potassium Salt of 5-chloro-4-hydroxy-1-methyl-2-oxo-N-phenyl-quinoline-3-carboxamide

5-chloro-4-hydroxy-1-methyl-2-oxo-N-phenyl-quinoline-3-carboxamide free acid (300 mg) was suspended in ethanol (6.0 mL) and 5 M aqueous potassium hydroxide (0.198 mL, ~ 1.1 eq) was added. The resulting suspension was shaken well by hand and then agitated and temperature-cycled between 40° C. and ambient for 48 hours.

The product was isolated by filtration, washed with ethanol (2 × 1 mL) and dried at 45° C. under vacuum to constant weight. White crystals were obtained. The yield was 251 mg.

### Example 2: Crystallisation of 5-chloro-4-hydroxy-1-methyl-2-oxo-N-phenylquinoline-3-carboxamide Potassium Salt (Example Compound 1) from a Variety of Solvents

5-chloro-4-hydroxy-1-methyl-2-oxo-N-phenyl-quinoline-3-carboxamide free acid (20 mg) was suspended in the solvent (400 µL) and 5 M aqueous potassium hydroxide (~ 1.1 eq) was added. The mixtures were shaken thoroughly by hand then temperature-cycled between ambient and 40° C. for at least 18-24 hours. The solid product was isolated by filtration.

The solvents investigated were as follows.

Ethanol
Acetone
Ethyl acetate
Acetonitrile
THF
Toluene
Methanol/water 50:50
Iso-propanol/water 90:10

In each case a crystalline salt was formed. All solids were analysed by X-ray Powder Diffraction analysis (XRPD). The same polymorphic form was obtained when the salt was isolated from each of the solvents investigated.

### Example 3: Characterisation of 5-chloro-4-hydroxy-1-methyl-2-oxo-N-phenylquinoline-3-carboxamide Potassium Salt (Example Compound 1)

### A) Solution NMR

A sample of Example compound 1 was dissolved in DMSO-d₆ and analysed by ¹H NMR (400 MHz). Peaks were seen at: δ: 13.09 (1H, s), 7.65 (2H, m), 7.35 (1H, m), 7.24 (3H, m), 7.05 (1H, m), 6.81 (1H, m), 3.49 (s, 3H).

The changes in the splitting pattern compared with the free API confirmed salt formation.

### B) Elemental Analysis

Elemental analysis was carried out as follows: for the determination of CHN a “Vario Micro Cube” (Elementar) was used. Chlorine content was determined using a Metrohm Model 883 Plus ion chromatograph. The potassium content was determined using an ICP-OES Model Spectro Arcos (Spectro). The analysis was carried out on duplicate samples of Example Compound 1.

The results of the elemental analysis were as follows:

| Atom | Predicted value | Measured value* |
|---|---|---|
| C | 55.7% | 55.3% |
| H | 3.3% | 3.4% |
| N | 7.6% | 7.5% |
| O | 13.1% | Not measured, but calculated to 13.6% |
| Cl | 9.7% | 9.6% |
| K | 10.7% | 10.6% |

* The figures presented in the table are the averages from two analytical runs.

The predicted values indicated in the table are the expected values for the monopotassium salt with no water of hydration. As is seen in the table, the potassium levels were found to be 10.6% w/w on average from the two runs. These results were consistent with a mono potassium salt with no water of hydration. (Theoretical 10.7%).

### C) Thermogravimetric Analysis (TGA) and Differential Scanning Calorimetry (DSC)

For the TGA analysis, approximately 5-10 mg of sample of Example Compound 1 was accurately weighed into a pierced Tzero aluminium pan and sealed with a pinhole lid using a crimper. The sample was then loaded into the chamber of a TGA thermal analyser at ambient temperature. A TA Instruments TGA5500 device was used. The sample was then heated at a rate of 20° C./min from 20° C. to 400° C. The purge gas used was nitrogen at a flow rate of 50 cm³/min.

For the DSC analysis, approximately 2 mg of sample was accurately weighed into a pierced Tzero aluminium pan and sealed using a universal crimper. The sample was then loaded into the chamber of a DSC analyser at ambient temperature. A TA Instruments DSC2500 device was used. The sample was then heated at a rate of 20° C./min from 30° C. to 400° C. The purge gas used was nitrogen at a flow rate of 50 cm³/min

A DSC trace for Example compound 1 is shown in FIG. 1. The numerical findings were as follows. The findings for the free acid compound are shown in the third column for comparison:

| | | |
|---|---|---|
| | Example Compound 1 | Free Acid |
| Solvent loss | None | ~0.1% |
| Degradation temperature | 347° C. ±2° C. | 260° C. ±2° C. |
| Melting point | 347° C. ±2° C. | 178° C. ±2° C. |
| Enthalpy of melting | 43.6 J/g | 91.9 J/g |

The STA data showed no sharp weight loss corresponding to an observable endotherm. A small weight loss of just under 1% at nearly 300° C. may have corresponded to some moisture trapped within the structure. This indicated the sample was not hydrated or solvated however. The onset of decomposition corresponded to the melt with onset at ~ 347° C. The TGA data showed a small gradual weight loss of 0.6% from the outset corresponding to surface moisture. From the DSC data, there was no significant endotherm before the melt with onset at ~ 343° C. The maximum endothermic heat flow was seen at 361.3° C.

Overall, these data demonstrate that the Example compound 1 was not hydrated and had good stability up to the temperature of 347° C.

### D) X-Ray Powder Diffraction Analysis (XRPD)

Approximately 5-10 mg of a sample of Example compound 1 was gently compressed on the XRPD zero background single obliquely cut silica sample holder. The sample was then loaded into a Philips X-Pert PRO diffractometer and analysed using the following experimental conditions:

| | |
|---|---|
| Scan Axis | Gonio |
| Start Position [°2θ] | 5.0084 |
| End Position [°2θ] | 49.9904 |
| Step Size [°2θ] | 0.0170 |
| Scan Step Time [s] | 1.9050 |
| Scan Type | Continuous |
| PSD Mode | Scanning |
| PSD Length [°2θ] | 2.12 |
| Offset [°2θ] | 0.0000 |
| Divergence Slit Type | Automatic |
| Irradiated Length [mm] | 10.00 |
| Specimen Length [mm] | 10.00 |
| Measurement Temperature [°C] | 25.00 |
| Anode Material | Cu |
| K-Alpha1 [Å] | 1.54060 |
| K-Alpha2 [Å] | 1.54443 |
| K-Beta [Å] | 1.39225 |
| K-A2 / K-A1 Ratio | 0.50000 |
| Generator Settings | 40 mA, 40 kV |
| Diffractometer Type | 0000000011038600 |
| Diffractometer Number | 0 |
| Goniometer Radius [mm] | 240.00 |
| Dist. Focus-Diverg. Slit [mm] | 100.00 |
| Incident Beam Monochromator | No |
| Spinning | Yes |

For certain repeat experiments, a slower scan speed was also used over a range of 4-40° 2theta as detailed below:

| | |
|---|---|
| Scan Axis | Gonio |
| Start Position [°2θ] | 4.0084 |
| End Position [°2θ] | 39.9804 |
| Step Size [°2θ] | 0.0170 |
| Scan Step Time [s] | 10.1600 |
| Scan Type | Continuous |
| PSD Mode | Scanning |
| PSD Length [°2θ] | 2.12 |
| Offset [°2θ] | 0.0000 |
| Divergence Slit Type | Automatic |
| Irradiated Length [mm] | 10.00 |
| Specimen Length [mm] | 10.00 |
| Measurement Temperature [°C] | 25.00 |
| Anode Material | Cu |
| K-Alpha1 [Å] | 1.54060 |
| K-Alpha2 [Å] | 1.54443 |
| K-Beta [Å] | 1.39225 |
| K-A2 / K-A1 Ratio | 0.50000 |
| Generator Settings | 40 mA, 40 kV |
| Diffractometer Type | 0000000011038600 |
| Diffractometer Number 0 | |
| Goniometer Radius [mm] | 240.00 |
| Dist. Focus-Diverg. Slit [mm] | 100.00 |
| Incident Beam Monochromator | No |
| Spinning | Yes |

The sample was confirmed by XRPD to be crystalline. The XRPD trace is shown in FIG. 2.

The peaks in the XRPD trace were as follows:

| Position (°2theta) | Height (Counts) | FWHM Left (°2theta) | D Spacing (Å) | Intensity (%) |
|---|---|---|---|---|
| 6.9268 | 2878.63 | 0.1004 | 12.76158 | 100 |
| 9.0221 | 104.08 | 0.1004 | 9.80198 | 3.62 |
| 11.9045 | 139.17 | 0.1004 | 7.4343 | 4.83 |
| 13.8896 | 191.53 | 0.1171 | 6.37596 | 6.65 |
| 15.6065 | 543.42 | 0.1171 | 5.67819 | 18.88 |
| 17.7674 | 188.33 | 0.1004 | 4.99216 | 6.54 |
| 18.3015 | 30.35 | 0.1673 | 4.84767 | 1.05 |
| 20.3398 | 162.03 | 0.1004 | 4.36623 | 5.63 |
| 20.8979 | 264.57 | 0.1506 | 4.25087 | 9.19 |
| 23.2972 | 60.21 | 0.1004 | 3.81824 | 2.09 |
| 23.5836 | 200.04 | 0.1171 | 3.77253 | 6.95 |
| 24.1885 | 262.45 | 0.1338 | 3.67953 | 9.12 |
| 24.9349 | 382.33 | 0.1004 | 3.57105 | 13.28 |
| 25.186 | 312.01 | 0.1004 | 3.53602 | 10.84 |
| 25.591 | 273.31 | 0.1004 | 3.48098 | 9.49 |
| 26.1304 | 103.7 | 0.1673 | 3.41032 | 3.6 |
| 26.8388 | 205.01 | 0.1338 | 3.3219 | 7.12 |
| 27.2575 | 314.61 | 0.1171 | 3.27182 | 10.93 |
| 27.9818 | 164.25 | 0.1004 | 3.18875 | 5.71 |
| 28.379 | 457.66 | 0.1506 | 3.14502 | 15.9 |
| 29.2124 | 252.55 | 0.1004 | 3.05716 | 8.77 |
| 29.7516 | 118.42 | 0.1338 | 3.00298 | 4.11 |
| 30.0826 | 93.35 | 0.1004 | 2.97068 | 3.24 |
| 30.3753 | 105.66 | 0.1673 | 2.94272 | 3.67 |
| 31.7158 | 141.46 | 0.1338 | 2.82134 | 4.91 |
| 32.1597 | 153.11 | 0.2007 | 2.7834 | 5.32 |
| 33.55 | 109.09 | 0.1338 | 2.67117 | 3.79 |
| 33.9854 | 196.54 | 0.1338 | 2.63794 | 6.83 |
| 34.481 | 79.86 | 0.1673 | 2.60115 | 2.77 |
| 35.2431 | 46.28 | 0.2007 | 2.54663 | 1.61 |
| 35.8148 | 66.33 | 0.2007 | 2.50728 | 2.3 |
| 36.5031 | 80.53 | 0.4015 | 2.46157 | 2.8 |
| 38.2021 | 68.3 | 0.1338 | 2.35591 | 2.37 |
| 39.2558 | 139.93 | 0.1673 | 2.29507 | 4.86 |
| 39.5981 | 137 | 0.1338 | 2.27602 | 4.76 |

When a sample of Example compound 1 was stored in a slurry in water at 20 mg / 400uL for 48 hours at ambient temperature, recovered, dried by evaporation and re-examined by XRPD, there was no change in the X-ray diffractogram. This indicates that the salt does not demonstrate a tendency to disproportionate.

### E) Optical Microscopy

Crystals of Example compound 1 were observed under an optical microscope and compared with crystals of the free acid compound 5-chloro-4-hydroxy-1-methyl-2-oxo-N-phenyl-quinoline-3-carboxamide (described in Example 1a) above).

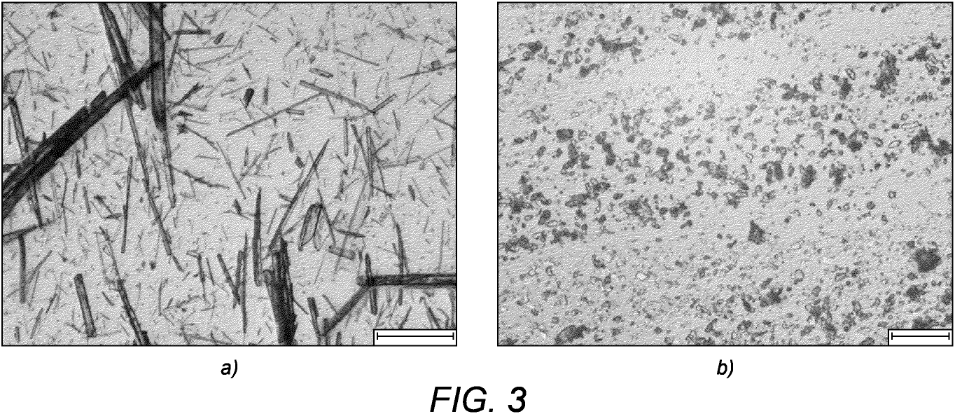

A photograph of the crystals of 5-chloro-4-hydroxy-1-methyl-2-oxo-N-phenylquinoline-3-carboxamide is shown in FIG. 3a. The crystals are seen to be large needles of length 200-300 microns. The bar on the photograph indicates the length 100 µm.

A photograph of the crystals of Example compound 1 is shown in FIG. 3b. The crystals are seen to be plate-like with a Particle Size Distribution of less than 10 microns. The bar on the photograph indicates the length 20 µm.

The free acid compound is seen to agglomerate, and this was visible with the naked eye. On the other hand, the potassium salt was found to be powder like with a plate like habit. That material is easier to handle than the long needles which made up the free acid compound.

### F) Solubility and Stability in Aqueous Solutions

The solubility and stability at room temperature of Example compound 1 and the free acid compound 5-chloro-4-hydroxy-1-methyl-2-oxo-N-phenyl-quinoline-3-carboxamide (described in Example 1a) above) was determined in PBS (pH 7.4) and in simulated colonic fluid (SCF) at ambient temperature. The SCF was the product FaSSCof obtained from Biorelevant.com prepared according to the manufacturer’s instructions. FaSSCof contains 0.15 mM sodium cholate, 0.3 mM Phospholipids, 0.1 mM Oleate, 120 mM sodium hydroxide, 45 mM TRIS and 76 mM maleate.

Saturated solutions were prepared in these solvents and the concentrations/impurity profile was determined by HPLC-UV using a calibration curve in the range of 1 to 150 µg/ml at 300 nm or 320 nm. Visual evaluation was also performed after every step over the testing period.

| | | |
|---|---|---|
| | PBS (pH7.4) | SFC |
| 5-chloro-4-hydroxy-1-methyl-2-oxo-N-phenyl-quinoline-3-carboxamide free acid | Non quantifiable | 3.44 µg/ml |
| 5-chloro-4-hydroxy-1-methyl-2-oxo-N-phenyl-quinoline-3-carboxamide potassium salt | 0.91 µg/ml | 7.99 µg/ml |

As can be seen in the table, Example compound 1 has better solubility in both of these solvents than the corresponding free acid.

The samples were also analysed for the presence of impurities. After a period of 48 and 72 hours, all impurities were below the lower limit of quantitation.

### G) Hygroscopicity

Dynamic Vapour Sorption (DVS) analysis was carried out on a sample of Example compound 1.

Approximately 20 mg of sample was placed into a wire-mesh vapour sorption balance pan and loaded into an ‘IgaSorp’ vapour sorption balance (Hiden Analytical Instruments). The sample was then dried by maintaining a 0% humidity environment until no further weight change was recorded.

The sample was then subjected to a step profile from 0 to 90% RH at 10% RH increments. The desorption isotherm was from 90% RH o 0% RH in 10% RH steps. The weight change during the sorption/desorption cycles were then monitored, allowing for the hygroscopic nature of the sample to be determined. Analysis was performed using the following parameters:
Analysis Mode F1
Wait Time 99%
Sample Temperature 25° C.
Min Time Out 30 Mins
MaxTime Out 180 Mins
Gas Flow Rate 250 ml/min

The results indicated that the sample was non-hygroscopic with a small reversible weight gain of less than 0.2% when analysed to 80% RH.

### Example 3: Biological Activity

### Example 3 A): Activity of 5-chloro-4-hydroxy-1-methyl-2-oxo-N-phenylquinoline-3-carboxamide Potassium Salt in Inhibiting Inflammation/Edema Associated with Inflammatory Bowel Disease

The efficacy of the 5-chloro-4-hydroxy-1-methyl-2-oxo-N-phenyl-quinoline-3-carboxamide potassium salt in inhibiting inflammation/edema associated with inflammatory bowel disease was evaluated in a CD4+ adoptive transfer induced inflammatory bowel disease in mice.

The details of the mice were as follows:

| | |
|---|---|
| Species/Strain or Breed: | Fox Chase C.B-17 SCID & Balb/C |
| Vendor: | Charles River |
| Age/Wt at Arrival: | CB-17 SCID — 6-7 weeks old Balb/C — 11-12 weeks old |
| Gender: | Female |
| Acclimation: | Acclimatization for at least 7 days after arrival |
| Housing: | 5 animals/cage |

On study day -1, SCID mice were weighed and evenly distributed into treatment groups based on body weight.

On study day 0, Balb/C mice were terminated, and spleens obtained for CD4⁺CD45RB^{high} cell isolation (Using SCID IBD Cell Separation Protocol). After cells were sorted and obtained, each animal in the treatment groups received an IP injection of CD4⁺CD45RB^{high} cells at a minimum 4x10⁵ cells (200 µl/mouse injections). A naive group of mice was followed through the experiment without receiving the injection of cells. The naive group comprised 5 animals.

On study day 21, treatment with 5-chloro-4-hydroxy-1-methyl-2-oxo-N-phenylquinoline-3-carboxamide potassium salt (1 mg/kg, daily day 21 to 49) was initiated. The compound was formulated as a 0.1 mg/mL suspension with sodium carboxymethyl cellulose (1%, w/v). Mice received either the 5-chloro-4-hydroxy-1-methyl-2-oxo-N-phenyl-quinoline-3-carboxamide potassium salt (Example compound 1) or vehicle. Each of those groups comprised 10 animals.

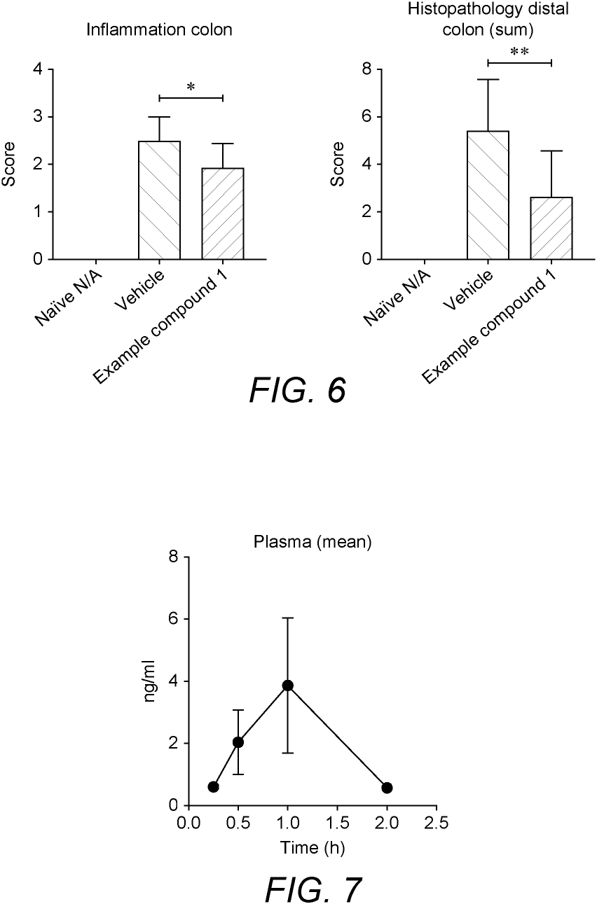

On study day 49, animals were anesthetized with Isoflurane and bled to exsanguination followed by cervical dislocation. The entire colon was removed, measured, and weighed. The colon was analysed for its level of interferon-γ and IL-22. The results are shown in FIG. 5. The inflammation in the colons was also scored by visual and histopathological assessments. The scores are shown in FIG. 6.

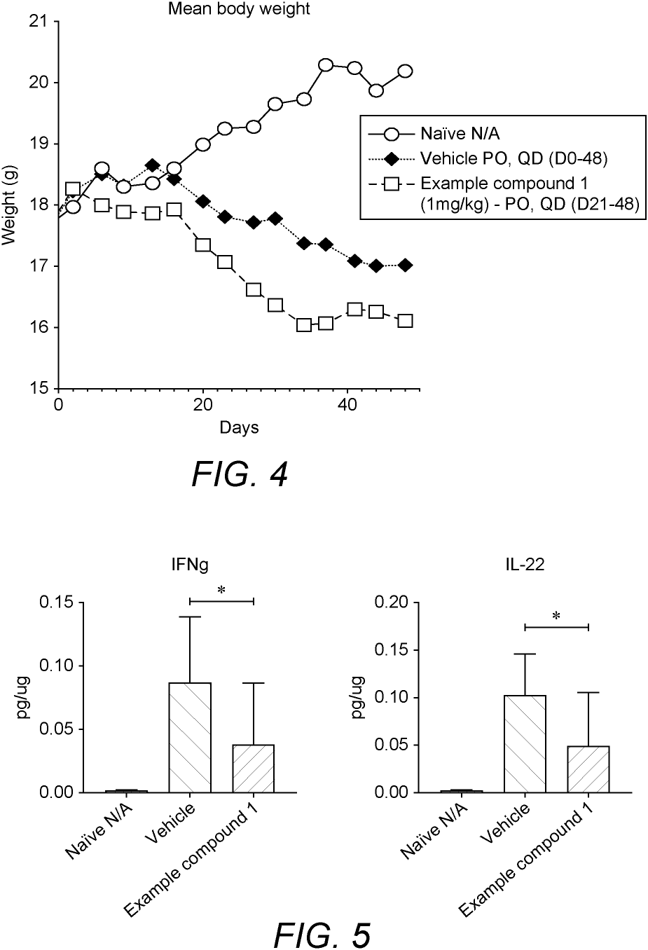

Each animal was weighed at 3- or 4-day intervals and the mean body weight of the mice in the three groups is shown in FIG. 4. As can be seen in the figure, the mice treated with Example compound 1 had less weight loss than mice treated with vehicle only. In FIG. 5, it is seen that the mice treated with Example compound 1 had lower levels of inflammatory markers than the mice treated with vehicle only. Similarly, it is seen in FIG. 6 that the mice treated with Example compound 1 had fewer signs of inflammation than the mice treated with vehicle only. (* indicates a statistical significance of p < 0.05; ** indicates a statistical significance of p < 0.01). The results in FIGS. 5 and 6 demonstrate that Example compound 1 has a local antiinflammatory effect in the colon of the animals.

### Example 3 B): Activation of the Aryl Hydrocarbon Receptor (AhR) in the Colon of Mice Following Administration of 5-chloro-4-hydroxy-1-methyl-2-oxo-N-phenylquinoline-3-carboxamide Potassium Salt

The potential of the 5-chloro-4-hydroxy-1-methyl-2-oxo-N-phenyl-quinoline-3-carboxamide potassium salt (Example compound 1) to activate the aryl hydrocarbon receptor (AhR) in the colon following oral administration was evaluated in a wild-type (WT) mice.

The details of the mice were as follows:

| | |
|---|---|
| Number of animals: | Order 51 (50 Study + 1 Extra) |
| Species/Strain or Breed: | C57Bl/6 |
| Vendor: | Taconic |
| Age/Wt at Arrival: | 6-7 weeks |
| Gender: | Female |
| Housing: | 5 animals/cage |

On study day 0, animals were weighed and evenly distributed into treatment groups based on body weight.

Also, on study day 0, treatments were initiated (a summary of the treatment schedule is shown in the table below). On study day 14, animals were asphyxiated via CO₂ inhalation followed by cervical dislocation. The entire colon was removed, collected and prepared for qPCR analysis of CYP1A1 (Normalized to GAPDH & ACTB).

| Group | N¹ | Treatment² | Dose Level (mg/kg) | Dose Route 3 | Regimen 4 | Dosing Days | Dose Vol (ml/kg) | Dose Conc (mg/ml) |
|---|---|---|---|---|---|---|---|---|
| 1 | 4 | Naive | N/A | N/A | N/A | N/A | N/A | N/A |
| 2 | 4 | Ex 1 | 1.0 mg/kg | PO | QD | D0-13 | 10 | 0.1 |
| 3 | 4 | Ex 1 | 0.1 mg/kg | PO | QD | D0-13 | 10 | 0.01 |
| 4 | 4 | Ex 1 | 0.01 mg/kg | PO | QD | D0-13 | 10 | 0.001 |

1: N, number of mice in the group.
2: Naive mice received no test compound or vehicle. Ex 1 = Example compound 1.
3: PO, per os (oral administration).
4: QD, quaque die (once a day).

The Example compound 1 was formulated as a 0.1 mg/ml, 0.01 mg/ml or 0.001 mg/ml suspension with sodium carboxymethyl cellulose (1%, w/v).

CYP1A1 qPCR results

| Group | Dose (mg/kg) | Colon CYP1A1 (fold change over untreated) | | Liver CYP1A1 (fold change over untreated) | |
|---|---|---|---|---|---|
| | | Mean | SE | Mean | SE |
| 2 | 1.0 | 8.34 | 2.75 | 3.44 | 0.30 |
| 3 | 0.1 | 7.42 | 2.62 | 2.81 | 0.65 |
| 4 | 0.01 | 1.03 | 0.34 | 1.08 | 0.34 |

As can be seen in the table WT mice treated with the Example compound 1 salt at 1.0 and 0.1 mg/kg had a significant increase in CYP1A1 mRNA expression compared to untreated animals indicating AhR activation. Furthermore, the results demonstrate that the Example compound 1 has a local AhR activating effect as the increase in liver CYP1A1 expression was lower than the increase in CYP1A1 in the colon.

### Example 3 C): In Vivo Pharmacokinetics of 5-chloro-4-hydroxy-1-methyl-2-oxo-N-phenyl-quinoline-3-carboxamide Potassium Salt

An in vivo pharmacokinetic study in rats was performed to determine if 5-chloro-4-hydroxy-1-methyl-2-oxo-N-phenyl-quinoline-3-carboxamide potassium salt (example compound 1) can be absorbed and detected systemically after oral dosing.

4 male Sprague Dawley rats, approximately 225 g - 250 g at dosing were dosed with 1 mg/kg of Example compound 2 formulated as a 0.1 mg/mL suspension with sodium carboxymethyl cellulose (1%, w/v). Blood samples were taken at 15 min, 30 min, 1 h, 2 h, 4 h, 6 h, 8 hand 24 h post dose

At each blood sampling approximately 250 µL blood was sampled in K3EDTA vials and approximately 100 µL plasma was also prepared.

The plasma samples were prepared by mixing 50 µL plasma with 250 µL of internal standard solution (20 ng/ml of phenacetin in ACN with 1% formic acid), mixed and centrifuged (20 min, 4000 rpm).

Plasma samples were transferred to Waters Ostro 96-well plate and drawn through the plate by applying 6-8 psi positive pressure for 10 min. 100 µL of supernatant was further diluted with 50 µL UP water and sample was submitted to analysis.

The standard and QC samples were prepared into blank rat colon homogenate and blank rat plasma. Standards were spiked into concentrations 0.1 - 10000 ng/ml of the analytes, QC samples into 3, 30, 300 and 3000 ng/ml, and were otherwise treated as samples.

The level of Example compound 1 in the plasma at the various time points is shown in FIG. 7. It is seen that the compound is detected in the plasma and that it is rapidly cleared.

### Example 3 D): Activity of 5-chloro-4-hydroxy-1-methyl-2-oxo-N-phenylquinoline-3-carboxamide in an Ulcerative Colitis Mouse Model

Ulcerative Colitis was modelled in C57B1/6 mice by administering Dextran Sulphate Sodium (DSS) in drinking water (1.5% w/v) for 5 days. Mice were monitored daily for weight loss and clinical symptoms of disease for 10 days starting at the beginning of DSS administration.

Animals in different treatment groups were administered either vehicle, 5-chloro-4-hydroxy-1-methyl-2-oxo-N-phenyl-quinoline-3-carboxamide free acid (1 mg/kg as a 0.1 mg/mL aqueous suspension with CMC—Na (sodium carboxymethyl cellulose, 2%, w/v), or anti-TNFα antibody (anti-mouse TNFα antibody clone XT3.11). One control group of mice did not receive DSS, 5-chloro-4-hydroxy-1-methyl-2-oxo-N-phenylquinoline-3-carboxamide free acid or vehicle (herein referred to as “naïve animals”). Vehicle and 5-chloro-4-hydroxy-1-methyl-2-oxo-N-phenyl-quinoline-3-carboxamide free acid were administered as daily administrations starting on either Day -7, though day 9. Anti-TNFα antibody was administered at 500 µg/treatment on Days 0, 2, 4, and 6. Following termination on Day 10, colons were removed and measured. The length and weight of the colon were also assessed.

Compared to naive animals, animals given DSS water developed clinical signs of disease including weight loss and diarrhoea as well as gross pathological signs of disease on experiment termination. Additionally, colon length was significantly reduced in diseased animals compared to naive animals, while weight was increased. Shortened length and increased weight:length ratio indicates edema associated with ulcerative colitis.

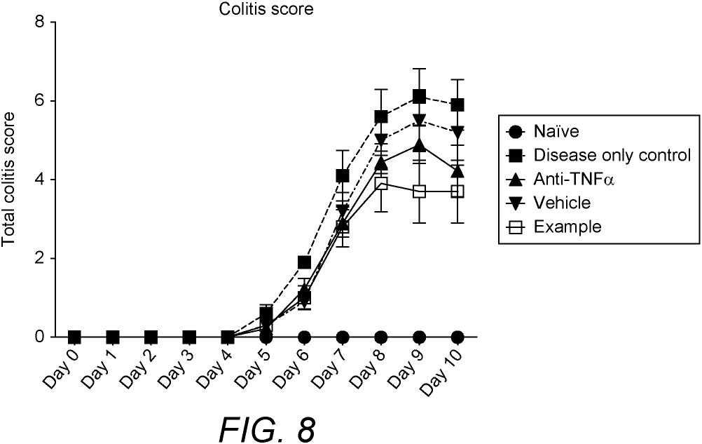

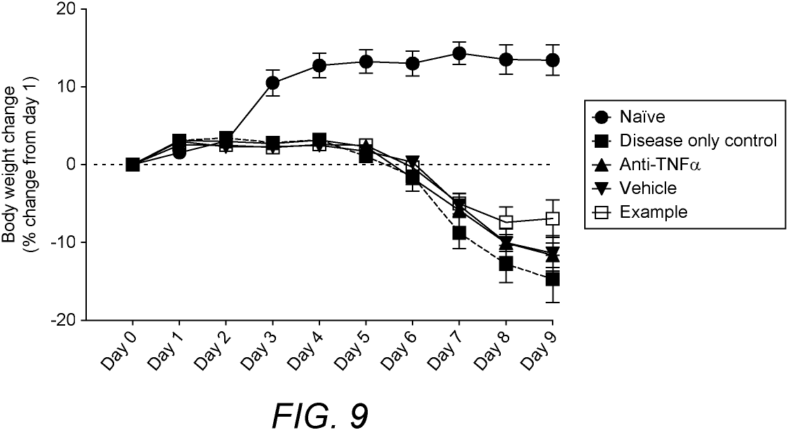

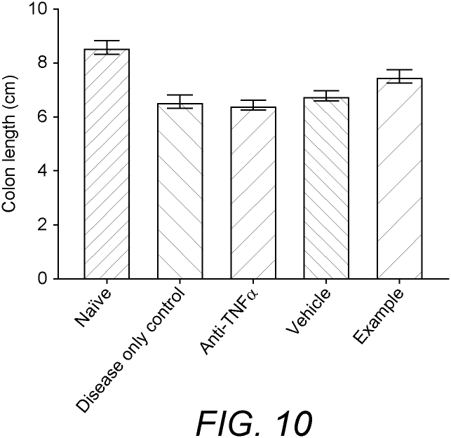

As shown in FIGS. 8 to 10, daily oral treatment with 5-chloro-4-hydroxy-1-methyl-2-oxo-N-phenyl-quinoline-3-carboxamide free acid (termed “Example” on FIGS. 8 to 10) resulted in a significant reduction in colitis clinical score on Day 9 of the study, reduced weight loss, and a significant increase in colon length on experiment termination compared to vehicle-treated controls, indicating an efficacious effect of the treatment.

### Example 4: 5-chloro-4-hydroxy-1-methyl-2-oxo-N-phenyl-quinoline-3-carboxamide Sodium Salt (Example Compound 2)

5-chloro-4-hydroxy-1-methyl-2-oxo-N-phenyl-quinoline-3-carboxamide free acid (300 mg) was suspended in ethanol (6.0 mL) and 5 M aqueous sodium hydroxide (0.198 mL, ~ 1.1eq) was added. The resulting suspension was shaken well by hand and then agitated and temperature-cycled between 40° C. and ambient for 48 hours.

The product was isolated by filtration, washed with ethanol (2 x 1 mL) and dried at 45° C. under vacuum to constant weight. White crystals were obtained. The yield was 240 mg.

When 5-chloro-4-hydroxy-1-methyl-2-oxo-N-phenyl-quinoline-3-carboxamide sodium salt was analysed by Thermogravimetric Analysis (TGA) using the method described in Example 3c) above, the numerical findings were as follows. The findings for the free acid compound are shown in the third column for comparison:

| | | |
|---|---|---|
| | Example Compound 2 | Free Acid |
| Solvent loss | 3.10% | ~0.1% |
| Degradation temperature | 350° C. ±2° C. | 260° C. ±2° C. |
| Melting point | 356° C. ±2° C. | 178° C. ±2° C. |
| Enthalpy of melting | 127.2 J/g | 91.9 J/g |

Approximately 5-10 mg of a sample of Example compound 2 was gently compressed on the XRPD zero background single obliquely cut silica sample holder. The sample was then loaded into a Philips X-Pert PRO diffractometer and analysed using the experimental conditions described in Example 3d) above. The sample was confirmed by XRPD to be crystalline. The peaks in the XRPD trace were as follows:

| Position (°2theta) | Height (Counts) | FWHM Left (°2theta) | D Spacing (Å) | Intensity (%) |
|---|---|---|---|---|
| 6.8328 | 281.87 | 0.1338 | 12.93691 | 14.22 |
| 7.2389 | 1981.62 | 0.1004 | 12.21201 | 100 |
| 9.2862 | 444.65 | 0.1004 | 9.52376 | 22.44 |
| 12.1652 | 303.67 | 0.1004 | 7.27561 | 15.32 |
| 12.5428 | 129 | 0.1171 | 7.05738 | 6.51 |
| 14.0265 | 146.8 | 0.1004 | 6.31404 | 7.41 |
| 14.5257 | 147.2 | 0.1171 | 6.09816 | 7.43 |
| 15.9343 | 482.16 | 0.1171 | 5.5621 | 24.33 |
| 17.8342 | 139.16 | 0.1338 | 4.97362 | 7.02 |
| 18.9295 | 369.42 | 0.1171 | 4.68824 | 18.64 |
| 20.6215 | 229.52 | 0.1338 | 4.30723 | 11.58 |
| 21.8289 | 147.92 | 0.1338 | 4.07164 | 7.46 |
| 22.2121 | 324.68 | 0.1338 | 4.00227 | 16.38 |
| 23.0972 | 567.42 | 0.1338 | 3.85085 | 28.63 |
| 24.514 | 1306 | 0.1338 | 3.6314 | 65.91 |
| 25.246 | 516.35 | 0.2007 | 3.52775 | 26.06 |
| 26.3611 | 650.87 | 0.1004 | 3.38101 | 32.85 |
| 26.5476 | 709.76 | 0.1171 | 3.35767 | 35.82 |
| 28.2156 | 220.44 | 0.3346 | 3.16286 | 11.12 |
| 28.4739 | 173.46 | 0.2007 | 3.13475 | 8.75 |
| 29.6785 | 370.45 | 0.1004 | 3.0102 | 18.69 |
| 31.2139 | 66.59 | 0.2676 | 2.86554 | 3.36 |
| 32.7791 | 137.02 | 0.1338 | 2.7322 | 6.91 |
| 33.3137 | 86.59 | 0.2007 | 2.68958 | 4.37 |
| 36.725 | 126.52 | 0.2676 | 2.4472 | 6.38 |
| 38.1042 | 75.91 | 0.4015 | 2.36174 | 3.83 |

When a sample of Example compound 2 was stored in a slurry in water at 20 mg / 400 uL for 48 hours at ambient temperature, recovered, dried by evaporation and re-examined by XRPD, there were slight changes in the X-ray diffractogram. This indicates that the salt demonstrated a tendency to disproportionate, albeit a slow tendency.

Crystals of Example compound 2 were observed under an optical microscope and compared with crystals of the free acid compound 5-chloro-4-hydroxy-1-methyl-2-oxo-N-phenyl-quinoline-3-carboxamide (described in Example 1a) above).

5-Chloro-4-hydroxy-1-methyl-2-oxo-N-phenyl-quinoline-3-carboxamide sodium salt was found to be powder like with a plate like habit and a Particle Size Distribution of less than 20 microns. The material is easier to handle than the long needles which made up the free acid compound.

The hygroscopicity of Example compound 2 was analysed using the method descriebd above in Example 3 g). The results indicated that the sample was slightly hygroscopic with a small reversible weight gain of 1% when analysed to 80% RH.

### Example 5: 5-chloro-4-hydroxy-1-methyl-2-oxo-N-phenyl-quinoline-3-carboxamide Lithium Salt (Example Compound 3)

5-chloro-4-hydroxy-1-methyl-2-oxo-N-phenyl-quinoline-3-carboxamide free acid (300 mg) was suspended in acetone (6.0 mL) and a solution of lithium hydroxide (24 mg) in water (0.2 ml) was added. The resulting suspension was shaken well by hand and then agitated and temperature-cycled between 40° C. and ambient for 48 hours.

The product was isolated by filtration, washed with ethanol (2 x 1 mL) and dried at 45° C. under vacuum to constant weight. White crystals were obtained. The yield was 225 mg.

When 5-chloro-4-hydroxy-1-methyl-2-oxo-N-phenyl-quinoline-3-carboxamide lithium salt was analysed by Thermogravimetric Analysis (TGA) using the method described in Example 3c) above, the numerical findings were as follows. The findings for the free acid compound are shown in the third column for comparison:

| | | |
|---|---|---|
| | Example Compound 3 | Free Acid |
| Solvent loss | 3.30% | ~0.1% |
| Degradation temperature | 360° C. ±2° C. | 260° C. ±2° C. |
| Melting point | 362° C. ±2° C. | 178° C. ±2° C. |
| Enthalpy of melting | 198.8 J/g | 91.9 J/g |

Approximately 5-10 mg of a sample of Example compound 3 was gently compressed on the XRPD zero background single obliquely cut silica sample holder. The sample was then loaded into a Philips X-Pert PRO diffractometer and analysed using the experimental conditions described in Example 3d) above. The sample was confirmed by XRPD to be crystalline. The peaks in the XRPD trace were as follows:

| Position (°2theta) | Height (Counts) | FWHM Left (°2theta) | D Spacing (Å) | Intensity (%) |
|---|---|---|---|---|
| 6.6562 | 15486.22 | 0.1004 | 13.27973 | 100 |
| 10.3703 | 503.93 | 0.1338 | 8.53048 | 3.25 |
| 12.1749 | 40.05 | 0.1338 | 7.26979 | 0.26 |
| 12.8045 | 57.45 | 0.1004 | 6.91373 | 0.37 |
| 13.2942 | 83.48 | 0.2007 | 6.66017 | 0.54 |
| 13.5732 | 83.31 | 0.1171 | 6.52388 | 0.54 |
| 14.2177 | 124.07 | 0.1004 | 6.22956 | 0.8 |
| 15.3564 | 302.58 | 0.1004 | 5.7701 | 1.95 |
| 15.8452 | 572.77 | 0.1004 | 5.59317 | 3.7 |
| 17.551 | 24.32 | 0.1004 | 5.05323 | 0.16 |
| 18.5711 | 269.05 | 0.1338 | 4.77789 | 1.74 |
| 19.0908 | 95.06 | 0.1004 | 4.64899 | 0.61 |
| 19.7568 | 348.64 | 0.1004 | 4.49375 | 2.25 |
| 20.0435 | 391.65 | 0.1004 | 4.43011 | 2.53 |
| 21.2321 | 911.12 | 0.1004 | 4.18471 | 5.88 |
| 22.1578 | 35.95 | 0.2007 | 4.01194 | 0.23 |
| 22.9311 | 203.45 | 0.1004 | 3.87837 | 1.31 |
| 23.1363 | 239.28 | 0.1004 | 3.84443 | 1.55 |
| 23.3393 | 158.88 | 0.1004 | 3.81146 | 1.03 |
| 24.3332 | 833.52 | 0.1004 | 3.65798 | 5.38 |
| 25.9287 | 1805.75 | 0.1506 | 3.4364 | 11.66 |
| 27.0238 | 662.61 | 0.1338 | 3.29957 | 4.28 |
| 27.833 | 120.46 | 0.1338 | 3.20545 | 0.78 |
| 28.6149 | 492.61 | 0.1004 | 3.11962 | 3.18 |
| 29.0144 | 83.12 | 0.1004 | 3.07757 | 0.54 |
| 30.0465 | 190.4 | 0.1171 | 2.97416 | 1.23 |
| 30.5601 | 368.53 | 0.1171 | 2.92535 | 2.38 |
| 30.9463 | 106.69 | 0.1004 | 2.88971 | 0.69 |
| 31.919 | 134.5 | 0.1338 | 2.80384 | 0.87 |
| 32.6937 | 87.54 | 0.1338 | 2.73915 | 0.57 |
| 33.3583 | 124.91 | 0.1338 | 2.68608 | 0.81 |
| 34.0206 | 156.78 | 0.1338 | 2.63529 | 1.01 |
| 35.0539 | 85.02 | 0.4015 | 2.55994 | 0.55 |
| 36.6066 | 124.18 | 0.1338 | 2.45484 | 0.8 |
| 37.709 | 98.6 | 0.2007 | 2.38557 | 0.64 |
| 39.2278 | 113.04 | 0.2342 | 2.29664 | 0.73 |

When a sample of Example compound 3 was stored in a slurry in water at 20 mg / 400uL for 48 hours at ambient temperature, recovered, dried by evaporation and re-examined by XRPD, there were slight changes in the X-ray diffractogram. This indicates that the salt demonstrated a tendency to disproportionate, albeit a slow tendency.

Crystals of Example compound 3 were observed under an optical microscope and compared with crystals of the free acid compound 5-chloro-4-hydroxy-1-methyl-2-oxo-N-phenyl-quinoline-3-carboxamide (described in Example 1a) above).

5-chloro-4-hydroxy-1-methyl-2-oxo-N-phenyl-quinoline-3-carboxamide lithium salt was found to be powder like with a plate like habit and a Particle Size Distribution of less than 20 microns. The material is easier to handle than the long needles which made up the free acid compound.

The hygroscopicity of Example compound 3 was analysed using the method described above in Example 3 g). The results indicated that the sample was slightly hygroscopic with a small reversible weight gain of less than 0.25% when analysed to 80% RH.

1. The compound 5-chloro-4-hydroxy-1-methyl-2-oxo-N-phenyl-quinoline-3-carboxamide potassium salt.

2. The compound as claimed in claim 1 in solid form.

3. The compound as claimed in claim 2 in crystalline form.

4. The compound as claimed in claim 3 which is a crystalline salt having a powder X-ray diffractogram with characteristic peaks at 2⊖ = 6.9±0.2°, 15.6±0.2°, 24.9±0.2° and 28.4±0.2°.

5. The compound as claimed in claim 4 wherein the powder X-ray diffractogram has further characteristic peaks at 2⊖ = 20.9°±0.2, 24.2±0.2°, 25.2±0.2°, 25.6±0.2° and 27.3±0.2°.

6. The compound as claimed in claim 3 wherein the crystalline form is characterized by a powder X-ray diffraction pattern in which the peak positions are substantially in accordance with the peak positions of the pattern shown in Figure 2.

7. The compound as claimed in claim 3 wherein the crystalline form is characterized by a differential scanning calorimetry trace substantially in accordance with that shown in Figure 1.

8. The compound as claimed in claim 2 in amorphous form.

9. The compound as claimed in claim 1 in aqueous solution form.

10. The compound as claimed in any one of claims 1 to 9 for use as a medicament.

11. The compound as claimed in any one of claims 1 to 9 for use as a medicament for the treatment or prophylaxis of an inflammatory bowel disease.

12. A pharmaceutical composition comprising a compound as claimed in any one of claims 1 to 9 and at least one pharmaceutically acceptable excipient.

13. The pharmaceutical composition as claimed in claim 12, further comprising one or more further therapeutic agents.

14. Use of the compound as claimed in any one of claims 1 to 9 in the manufacture of a medicament for the treatment or prophylaxis of an inflammatory bowel disease.

15. A method of treating or preventing an inflammatory bowel disease comprising a step of administering to a subject in need thereof a compound as claimed in any one of claims 1 to 9, or the pharmaceutical composition as claimed in any one of claims 12 or 13.

16. The method as claimed in claim 15, or the use as claimed in claim 14, wherein the inflammatory bowel disease is Crohn’s disease or Ulcerative Colitis.

17. The method as claimed in any one of claims 15 or 16, comprising a step of orally or rectally administering the compound or the pharmaceutical composition to the subject.
